(19) United States
(12) Patent Application Publication
(10) Pub. No.: US 2023/0295669 A1
(43) Pub. Date: Sep. 21, 2023

(54) INDUSTRIAL PROCESS FOR OBTAINING AN AGRICULTURAL COMPOSITION CONSTITUTED BY SOLUBILIZING AND PHOSPHORUS MINERALIZING MICROORGANISMS, AND USE IN THE PRODUCTION AND OPTIMIZATION OF MINERAL, ORGANOMINERAL AND ORGANIC FERTILIZERS

(71) Applicant: BIOTROP SOLUÇÕES BIOLÓGICAS E PARTICIPAÇÕES LTDA., Vinhedo (BR)

(72) Inventors: Josiane FUKAMI, Vinhedo (BR); Douglas Fabiano GOMES, Vinhedo (BR); Juliana Marcolino GOMES, Vinhedo (BR); Jonas HIPOLITO DE ASSIS FILHO, Vinhedo (BR)

(21) Appl. No.: 17/920,006

(22) PCT Filed: Oct. 26, 2021
(86) PCT No.: PCT/BR2021/050469

§ 371 Date: Oct. 19, 2022

(51) Int. Cl. C12P 3/00; C05B 7/00

(52) U.S. Cl. CPC C12P 3/00; C05B 7/00

(57) ABSTRACT

The present invention refers to an industrial process for obtaining an agricultural composition formed by association of one or more species of Bacillus spp., of Lactobacillus spp. and of Pseudomonas, the process for induction of exudates/metabolites, as well as the application thereof in the manufacture and in the increase of the efficiency of phosphate fertilizers of mineral, organomineral and organic origin. Surprisingly, the agricultural composition used in the fertilization industry increases the availability of macro and micronutrients to the plants with agricultural interest, such as soy and corn. Finally, both the use of the agricultural composition in the fertilizers industry as the application of the product in the field potentialize the greater availability of essential nutrients for the development of the cultures.

### FIELD OF THE INVENTION

The present invention refers to the industrial process for obtaining an agricultural composition and the application thereof in the manufacture and optimization of phosphate fertilizers of mineral, organomineral and organic origin, which uses different genus (Pseudomonas, Lactobacillus and Bacillus), as well as the induction of their exudates/metabolites, capable of solubilizing and mineralizing insoluble nutrients in soluble compounds, as well as their application in the field in the increase of the availability of macro and micronutrients to the plants of agricultural interest.

### BACKGROUND OF THE INVENTION

To supply the food demand required by the growing worldwide population, estimated in 9 billion people up to 2050, the agricultural production must increase significantly (Hasler et al., 2017; United Nations, 2015; Hazell and Wood, 2008). This increase is occurring, however not always with the use of sustainable practices, such as took place up to the 1990s, when the increase in production was, to a large extent, attributed to the indiscriminate use of inorganic fertilizers (Rahman and Zang 2018). Even nowadays, the exaggerated application of nitrogenated fertilizers in agricultural lands, which, added to the low efficiency of the material, leads to the accumulation of nitrates in the soil, contributing to the contamination of groundwater and to the global heating (Kool et al., 2011). Another problem associated with the excessive use of fertilizers is the deposition of heavy metals in the soils, whereby the cadmium and chromium are the most frequent (Savci, 2012). Therefore, the adoption of innovative agricultural technologies is fundamental to support the production of quality foods, and includes the application of efficient fertilization methods, since the unreasonable application of fertilizers results in environmental problems (Tilman et al., 2002; Pretty and Hine, 2011). In fact, the need to increase the productivity of the crops is necessary, however, it must be associated to sustainable agricultural practices, which guarantee the food and environmental security.

The fertilizers applied in agriculture are classified according to their nature and composition in three categories, mineral, organic and organomineral. The first, is constituted by inorganic compounds (absence of associated carbon) and are subdivided in simple or mixed, when they present only one nutrient or a complex of two or more, respectively. The organic fertilizers are constituted solely of organic matter, while the organominerals mix the organic matter with inorganic nutrient sources. Additionally, the fertilizers are used with the main purpose of providing and supplementing the essential macronutrients (nitrogen, potassium and phosphorus—NPK) for the vegetable development, being the most commonly applied via soil.

The phosphorus when absent or in insufficient quantities, limits the development and performance of cultures of agricultural interest. A key compound of the most important metabolic processes, including the photosynthesis, the transfer of energy and the biosynthesis of macromolecules, phosphorus must be acquired by the plants directly from the environment, which is made difficult by presenting low solubility (Richardson et al., 2009). Another characteristic which makes obtaining this macronutrient more difficult is related to its insoluble inorganic form, when it is presented immobilized in rocky compounds, adsorbed to minerals (iron phosphate, aluminum phosphate and calcium phosphate), making its assimilation more difficult by the plants. A more common phenomenon is tropical soils with high degree of weathering such as the Latosoils and Argisoils which correspond to 58% of the Brazilian soils (dos Santos et al., 2018) and rich in iron oxides and aluminum such as hematite. This leaves only 0.1% of all the phosphorus present in the environment being available for the nutrition of plants, which are capable of assimilating only the soluble forms, such as the phosphate ion.

Alternatively, since it is a resource that is not always available, in agricultural crops the phosphorus is supplemented by fertilizer, supplying the demand of the plants for this nutrient. Even in this manner, not all the phosphorus applied as fertilizer is used, part is lost by the leaching process, and may cause environmental impacts, or by means of the rapid mineralization of this nutrient, causing the efficiency of the fertilization to rarely exceed the 30% (Hemwall, 1957; Baligar and Bennett, 1986).

Apart from the fertilization, the use of microorganisms that are beneficial in agriculture is increasingly more frequent, aiming at different aspects such as promotion of growth, nutrient mobilization, as well as the control of pests and diseases.

The expression “plant growth-promoting bacteria (PGPB)” was used for the first time by Klopper and Schroth (1978) to describe bacteria of the soil which colonize the roots and/or rhizosphere of the plants and increase the growth thereof. These bacteria became extensively studied in the last years, generating important results on the mechanisms which perform and provide to the better development of the plants. Among the several genera of microorganisms characterized as PGPB there are noted Agrobacterium, Allorhizobium, Arthrobacter, Azospirillum, Azotobacter, Bacillus, Bradyrhizobium, Burkholderia, Caulobacter, Chromobacterium, Erwinia, Exiguobacterium, Flavobacterium, Mesorhizobium, Micrococcous, Providencia, Pseudomonas, Rhizobium and Serratia (Yadav et al., 2017; Suman et al., 2015; Suman et al., 2016).

The differentiation of the microorganisms as plant growth microorganisms is connected to the identification of one or more action mechanisms, with emphasis on phosphorus solubilization (Pikovskaya, 1948), zinc (Fasim et al., 2002) and potassium (Hu and Guo, 2006), the production of phytohormones such as auxins (Bric et al., 1991) and gibberellins (Brown, 1968), the biological fixation of the nitrogen (Boddey et al., 1995) and the production of the ACC-deaminase enzyme (Jacobson et al., 1994). Moreover, further attributes of the bacteria that benefit plants are the biosynthesis of ammonia (Cappucino and Sherman, 1992), HCN (Bakker and Schippers, 1997), siderophores (Schwyn and Neilands, 1987) and the antagonist action to phytopathogens.

To optimize the efficiency in obtaining the phosphorus, the microorganisms perform important functions in the phosphorus cycle, since they present the ability to make available this macronutrient to the plants. This group is subdivided between solubilizing and mineralizing. The great difference between mineralizing and solubilizing is related to the manner in which they make available the phosphorus to the plants, whether by means of the enzymatic action or by means of the synthesis of organic acids, respectively (Guang-Can et al., 2008). Therefore, the use of specific microorganisms in their induced forms, combining the enzyme biosynthesis and organic acids, in the industrial process of production of phosphate fertilizers, increases the efficiency of these products, reducing losses, and enabling the reduction of the dose and reducing the cost of the agricultural activity and the risk of causing damages to the environment, that is, promoting the sustainability of the agribusiness.

In this context, aiming at increasing the efficiency of the phosphate fertilization, we propose the application of microorganisms and the metabolites thereof in the industrial process of extraction and processing of rocks and minerals from which originate the fertilizer products that are rich in phosphorus. As will also be understood by a person skilled in the art we further propose the application thereof to the field in the increase of the availability of other macro and micronutrients to the plants with agricultural interest considering the several action mechanisms which the microorganisms perform in the crops.

### REFERENCES

Atikur Rahman, K. M.; Zhang, D. Effects of Fertilizer Broadcasting on the Excessive Use of Inorganic Fertilizers and Environmental Sustainability. Sustainability, 2018.
Bakker, A. W.; Schippers, B. Microbial cyanide production in the rhizosphere in relation to potato yield reduction and Pseudomonas SPP-mediated plant growth-stimulation. Soil Biol Biochem. v. 19, n. 4, p. 451-457, 1987.
Baligar, V. C., & Bennett, O. L. NPK—fertilizer efficiency—a situation analysis for the tropics. Fertilizer research, v. 10, n. 2, p. 147-164, 1986.
Bric, J. M.; Bostock, R. M.; Silverstone, S. E. Rapid in situ assay for indoleacetic acid production by bacteria immobilized on a nitrocellulose membrane. Appl Environ Microbiol. v. 57, n. 2, p. 535-538, 1991.
Boddey, R.; De Oliveira, O.; Urquiaga, S.; Reis, V.; De Olivares, F. et al. Biological nitrogen fixation associated with sugar cane and rice: contributions and prospects for improvement. Plant Soil. v. 174, n. 1-2, p. 195-209, 1995.
Brown, Peter H.; HO, Tuan-Hua David. Barley aleurone layers secrete a nuclease in response to gibberellic acid: purification and partial characterization of the associated ribonuclease, deoxyribonuclease, and 3′-nucleotidase activities. Plant Physiology, v. 82, n. 3, p. 801-806, 1986.
Cappucino, J. C.; Sherman, N. Nitrogen Cycle. In: Microbiology: A Laboratory Manual. (4th edn), Benjamin/Cumming Pub Co, New York, USA, p.311-312, 1992.
Fasim, F.; Ahmed, N; Parsons, R.; Gadd, G. M. Solubilization of zinc salts by a bacterium isolated from the air environment of a tannery. FEMS Microbiol Lett. v. 213, n. 1, p. 1-6, 2002.
Guang-Can, T. A. O., Shu-Jun, T. I. A. N., Miao-Ying, C. A. I., & Guang-Hui, X. I. E. Phosphate-solubilizing and -mineralizing abilities of bacteria isolated from soils. Pedosphere, v.18, n.4, p. 515-523, 2008.
Hasler, K.; Olfs, H.-W.; Omta, O.; Bröring, S. Drivers for the Adoption of Different Eco-Innovation Types in the Fertilizer Sector: A Review. Sustainability. v. 9, p.2216, 2017
Hu X, Chen J, Guo J. Two Phosphate- and Potassium-solubilizing Bacteria Isolated from Tianmu Mountain, Zhejiang, China. World J Microbiol Biotechnol. v. 22, n. 9, p. 983-990, 2006.
Hazell, P.; Wood, S. Drivers of change in global agriculture. Philos. Trans. R. Soc. B Biol. Sci. v. 363, p. 495-515, 2008.
Hemwall, J. B. The fixation of phosphorus by soils. Adv Agron v. 9, p. 95-112, 1957.
dos Santos, H. G.; Jacomine, P. T.; Dos Anjos, L. H. C.; De Oliveira, V. A.; Lumbreras, J. F.; Coelho, M. R.; Almeida, Araujo filho, J. C.; Oliveira, J. C.; Cunha, T. J. F. Brazilian Soil Classification System. Embrapa Solos-Livro técnico (INFOTECA-E), 2018.
Kloepper, J.; Schroth, M. Plant growth-promoting rhizobacteria on radishes. In: Proceedings of the 4th international conference on plant pathogenic bacteria, p 879-882, 1978.
Jacobson, C. B.; Pasternak, J.; Glick, B. R. Partial purification and characterization of 1-aminocyclopropane-1-carboxylate deaminase from the plant growth promoting rhizobacterium Pseudomonas putida GR12-2. Can J Microbiol v. 40, n. 12, 1019-1025, 1994.
Kool, D. M., Dolfing, J., Wrage, N., & Van Groenigen, J. W. Nitrifier denitrification as a distinct and significant source of nitrous oxide from soil. Soil Biology and Biochemistry, v. 43, n. 1, p. 174-178, 2011.
Pikovskaya, R. Mobilization of phosphorus in soil in connection with vital activity of some microbial species. Mikrobiologiya. v. 17, p. 362-370, 1948.
Pretty, J.; Hine, R. Reducing Food Poverty with Sustainable Agriculture: A Summary of New Evidence; University of Essex: Essex, U K, 2011.
Richardson, A. E., Barea, J. M., McNeill, A. M., & Prigent-Combaret, C. Acquisition of phosphorus and nitrogen in the rhizosphere and plant growth promotion by microorganisms. Plant and soil, v. 321, n. 1, p.305-339, 2009.
Schwyn, B.; Neilands, J. Universal chemical assay for the detection and determination of siderophores. Anal Biochem v. 160, n. 1, p. 47-56, 1987.
Serpil Savci. Investigation of Effect of Chemical Fertilizers on Environment. APCBEE Procedia. v. 1, p. 287-292, 2012.
Suman, A.; Verma, P.; Yadav, N. A.; Saxena, A. K. Bioprospecting for extracellular hydrolytic enzymes from culturable thermotolerant bacteria isolated from Manikaran thermal springs. Res J Biotechnol. v. 10, p. 33-42, 2015.
Suman, A; Verma, P.; Yadav, A. N.; Srinivasamurthy, R.; Singh, A.; Prasanna, R. Development of hydrogel-based bio-inoculant formulations and their impact on plant biometric parameters of wheat (Triticum aestivum L.). Int J Curr Microbiol Appl Sci. v. 5, n. 3, p. 890-901, 2016.
Swietlik, D. Causes and Consequences of Overfertilization in Orchards. Hort Technology. v. 2, p. 112-132, 1992.
Tilman, D.; Cassman, K. G.; Matson, P. A.; Naylor, R.; Polasky, S. Agricultural sustainability and intensive production practices. Nature. v. 418, p. 671-677, 2002.
United Nations. Key Findings and Advance Tables. In World Population Prospects: The 2015 Revision, ESA/P/WP.241; United Nations: New York, NY, USA, 2015.
Yadav, A. N.; Verma, P.; Kumar, V.; Sachan, S. G.; Saxena, A. K. Extreme Cold Environments: A Suitable Niche for Selection of Novel Psychrotrophic Microbes for Biotechnological Applications. Adv Biotechnol Microbiol. v. 2, n. 2, p. 1-4, 2017.

### SUMMARY OF THE INVENTION

In a general manner, one of the main industrial routes for obtaining phosphate fertilizers is by means of the exploration of natural phosphate deposits, considered as being a non-renewable source. The phosphate rocks are extracted in a mechanization process and attacked with sulfuric acid for the production of these fertilizers. Even going through this extraction process, part of the nutrients present in these fertilizers is not assimilated by the plants when applied to the field.

Thus, the present invention employs microorganisms of different genera (Pseudomonas, Lactobacillus and Bacillus), as well as their metabolites induced in a specific industrial process, capable of solubilizing and mineralizing insoluble phosphates in soluble phosphate compounds during the process of production of fertilizers.

The present invention further enables the treatment of the subproducts that are generated in the industrialization of the fertilizers, converting them into compounds with potential for agricultural application.

The applicability of the invention occurs, mainly for the optimization of phosphate fertilizers, and may be used in different steps of the productive process for obtaining the fertilizers, that is, it can be applied during the production or at the end of the process.

The present invention teaches that, surprisingly, it is possible to develop a biotechnological solution (in industrial scale) containing one or more species of Bacillus in their resistance form—endospores, one or more species of Lactobacillus and Pseudomonas, as well as their metabolites induced in a specific industrial process which are capable of solubilizing and mineralizing insoluble phosphates in soluble phosphate compounds during the process of production of fertilizers.

The present invention further provides an agricultural composition produced by the method of the present invention, as well as the use of the same in the fertilizer industry and in agriculture.

Advantageously, the present invention allows obtaining an agricultural composition which potentializes the efficiency of the phosphate fertilizers applied to the field for several cultivations of agronomical interest, such as soy bean, corn, wheat, rice, among others.

As will be understood by a person skilled in the art, the present invention provides additional parameters for the method of production of an agricultural composition formed by two or more species of Bacillus, Lactobacillus and Pseudomonas fermented in industrial scale, demonstrating the necessary parameters for the cell sporulation of species of Bacillus and induction of metabolites for the species of Lactobacillus and Pseudomonas, such as parameters of pressure, temperature, oxygenation (air volume and agitation) and culture medium, enabling obtaining a biotechnological product.

Advantageously, the organic acids produced via industrial induction of Pseudomonas and Lactobacillus act instantaneously on the inorganic phosphates during the manufacturing process of the phosphate fertilizers, while the Bacillus can act mainly when these phosphates are applied to the field, improving the availability of the plant absorption, since they produce a series of compounds that are capable of mineralizing the phosphorus contained in the fertilizers and release the adsorbed fraction to the soil colloids.

In a first embodiment, the present invention provides a production process of an agricultural composition comprising the steps of:
(a) fermenting the microorganisms comprising five species, which are Bacillus subtilis, B. licheniformis, Lactobacillus plantarum and L. buchneri and Pseudomonas fluorescens to obtain an agricultural composition with the induction of the metabolites capable of solubilizing and mineralizing the phosphorus, by means of the specific formulation for each microorganism during the industrial process; and
(b) formulation of a biotechnological product comprised by the bacteria mix, in a technical solution which allows the application in the phosphate fertilizer industry, as well as the residues thereof, to potentialize the solubilization of phosphorus making it promptly available to the plants.
(c) formulation of a biotechnological product comprised by the bacteria mix, in a technical solution which allows the application in agriculture to increase the availability of the macro and micronutrients to the plants with agricultural interest.

In a surprising manner, the present invention has as its preferred embodiment the potentializing of the mineralization of phosphorus. In a secondary embodiment, in an unexpected manner, the present invention is capable of increasing the solubilization of phosphorus of the fertilizers applied to the field in consequence of the viable microorganisms that are present in the fertilizers, according to the preferred embodiments of the use of these products in agriculture, which are broadcasting, sowing furrow with the phosphate fertilizer.

In an alternative embodiment, the present invention provides a biotechnological product which can also be applied directly in the cultivations with agronomic interest, preferably via seeds or sowing furrow.

### BRIEF DESCRIPTION OF THE FIGURES

For a more complete understanding of the invention, reference must be made now to the embodiments of the invention illustrated in more detail in the figures accompanied and described by means of the embodiments of the invention.

FIGS. 1A-1C illustrate the phosphorus analysis in a fertilizer sample during the production process (ground stage). A. P-CNA; B. P-Total; C. P-CNA/P-Total Conversion Rate. Biologic 1—Lactobacillus plantarum and Lactobacillus buchneri; Biologic 2—Pseudomonas fluorescens; Biologic 3—Lactobacillus plantarum, Lactobacillus buchneri and Pseudomonas fluorescens.

FIGS. 2A-2C illustrate the phosphorus analysis in fertilizer sample during the production process (final product—granulated). A. P-CNA; B. P-Total; C. P-CNA/P-Total Conversion Rate. Biologic 1—Lactobacillus plantarum and Lactobacillus buchneri; Biologic 2—Pseudomonas fluorescens; Biologic 3—Lactobacillus plantarum, Lactobacillus buchneri and Pseudomonas fluorescens; Biologic 4—Lactobacillus plantarum, Lactobacillus buchneri, Bacillus subtilis and Bacillus licheniformis.

FIGS. 3A-3C illustrate the phosphorus analysis in a fertilizer sample during the production process (process subproduct). A. P-CNA; B. P-Total; C. P-CNA/P-Total Conversion Rate. Biologic 1—Lactobacillus plantarum and Lactobacillus buchneri; Biologic 2—Pseudomonas fluorescens; Biologic 3—Lactobacillus plantarum, Lactobacillus buchneri and Pseudomonas fluorescens; Biologic 4—Lactobacillus plantarum, Lactobacillus bucnheri, Bacillus subtilis and Bacillus licheniformis.

FIG. 4 illustrates the average productivity of the soy bean culture carried out in 8 different regions. The treatments were inoculated with the microorganisms on their own or mixed and 25% reduction of phosphate fertilizing.

FIG. 5 illustrates the average productivity of the corn culture carried out in 8 different regions. The treatments were inoculated with the microorganisms on their own or mixed and 25% reduction of phosphate fertilizing.

### DETAILED DESCRIPTION OF THE INVENTION

In a preferred embodiment, according to the present invention, the fermentation (step (a)) of the different Bacillus, Lactobacillus and Pseudomonas by batch occurs for approximately 24-168 hours.

In a preferred embodiment, the method of the present invention comprises the sequencing expansion (scaling-up) of the culture of Bacillus, Lactobacillus and Pseudomonas for inoculation of the fermentation culture. Preferably, the sequencing expansion starts in volumes of 100 mL, which serves to inoculate 1 L. This, in its turn, is inoculated in 10 L, which, then are inoculated two balloons in 180 L tanks and which, finally, are transferred to reactors containing 2,000 L.

In a preferred embodiment, the species of Bacillus and Pseudomonas are expanded in 100 mL flasks by incubation in orbital shaker of 80 rpm to 200 rpm, and without shaking when cultivated the species of Lactobacillus. The incubation time is of, preferably, 8 hours to 48 hours. Preferably, the species of Bacillus are then cultivated in stainless-steel balloons containing 1 L of culture medium. The incubation time is preferably of 8 to 48 hours with air flow of 0.25 Nm³/h to 1.0 Nm³/h (=4.16-16.67 vvm). Preferably, the species of Pseudomonas are cultivated in flasks of around 1 L of culture medium by incubation in orbital shaker at 80 rpm to 200 rpm. Preferably the species of Lactobacillus are cultivated without shaking.

In a preferred embodiment, the air flow of the stainless-steel balloons containing 10 L for the cultivation containing the species Bacillus and Pseudomonas is of 0.25 to 1.5 Nm³/h (=0.41-2.5 vvm), and the incubation time is preferably of about 8 hours to about 48 hours.

In a preferred embodiment, stainless-steel balloons containing 10 L for the cultivation containing the species of Lactobacillus are cultivated without the need for aeration.

In a preferred embodiment, the incubation temperature for multiplication of the species of Bacillus, Lactobacillus and Pseudomonas according to the present invention is from 22° C. to 38° C.

In a preferred embodiment, the species of Bacillus, Lactobacillus and Pseudomonas are inoculated separately in the scaling-up process up to 180 L and mixed in the 2.000 L fermenters as described for the present invention. For this, in a preferred embodiment, after the cultivation of Bacillus in two stainless-steel balloons with 1 L of culture medium, the referred balloons are inoculated in two other stainless-steel balloons of 10 L and then transferred in tanks containing 180 L of specific culture medium for each microorganism, whereby Table 2 shows the specific culture medium for the B. licheniformis; and Table 3 the specific culture medium for the B. subtilis with the addition of a stainless-steel balloon containing 5 L of the Endospore formation inductor salt solution for the Bacillus spp. (Table 4), incubated for 24 to 168 hours. The air flow is, preferably of 1.0 to 15.0 Nm³/h (=0.16-1.25 vvm).

In a preferred embodiment, after the cultivation of two flasks containing 1 L of Lactobacillus and Pseudomonas, the referred cultivations are inoculated in two other stainless-steel balloons of 10 L and then transferred to tanks containing 180 L of specific culture medium for each microorganism, whereby Table 5 shows the specific culture medium for the species of Lactobacillus; and Table 6 for the specific culture medium for Pseudomonas, incubated for 24 to 168 hours. The air flow for the Pseudomonas is, preferably, from 1.0 to 15.0 Nm³/h (=0.16-1.25 vvm) and for the cultivation of the Lactobacillus there is no need for aeration during the incubation.

In a preferred embodiment, the step of mixing of the Bacillus, Lactobacillus and Pseudomonas is carried out with temperature from 22° C. to 38° C. The air flow is preferably of 1.0 Nm³/h a 2.5 Nm³/h (=0.0085-0.021 vvm). The pressure is preferably of 0.5 to 1.2 kgf/cm³. The shaking is preferably from 40 hz to 45 hz.

### EXAMPLES

### Example 1—Scaling Up of Culture

The different species of Bacillus, Pseudomonas and Lactobacillus are inoculated separately in flasks containing 100 mL of the culture medium as described in Table 1, 6 and 5, respectively, being incubated in orbital shaker of 80-200 rpm, at 22-38° C. for approximately 8-48 hours, with exception of the species of Lactobacillus which do not require shaking, only incubation at the temperature of 22-38° C. The next step in the scaling-up for the Bacillus consists in the inoculation of stainless-steel balloons containing 1 L of culture medium (Table 1), wherein the species are separately cultivated and incubated for approximately 8-48 hours, with air flow of 0.25-1.0 Nm³/h (=4.16-16.67 vvm) and temperature approximately of 22-38° C. For the Pseudomonas, the inoculum of 100 mL is then transferred to flasks containing 1 L of culture medium (Table 6), being incubated in orbital shaker of 80-200 rpm, at 22-38° C. for approximately 8-48 hours. In the same manner, 100 mL of each species of Lactobacillus are transferred to 1 L of culture medium (Table 5) and incubated at a temperature of 22-38° C. for approximately 8-48 hours.

After the incubation period, the species of Bacillus and Pseudomonas the cultivations are inoculated in stainless-steel balloons containing 10 L of specific culture medium for each microorganism and incubated for approximately 18-96 hours, with air flow 0.25-1.5 Nm³/h (=0.41-2.5 vvm) and temperature varying from 22-38° C. For the cultivation of Lactobacillus there is no need for aeration during the incubation time.

TABLE 1
CULTURE MEDIUM USED FOR THE GROWTH OF THE
BACILLUS SPP. UP TO THE SCALE-UP OF 10 L.

| | Reagents | | |
|---|---|---|---|
| 01 | K₂HPO₄ | 0.1-4 | g |
| 02 | KH₂PO₄ | 0.1-4 | g |
| 03 | MgSO₄•7H₂O | 0.1-0.6 | g |
| 04 | NaCl | 0.05-0.3 | g |
| 05 | Yeast Extract | 0.1-4 | g |
| 06 | Peptone | 0.2-4 | g |
| 07 | Solution FeCl₃ 10% | 0.05-1 | mL |
| 08 | Solution MnSO₄ 10% | 0.05-1 | mL |
| 09 | Saccharose | 5-10 | g |

| | | |
|---|---|---|
| 10 | Water | q.s.p. 1 L |

q.s.: quantum sufficit

After this time, each culture containing two stainless-steel balloons with 10 L of culture medium is inoculated in a tank containing 180 L of specific culture medium for each microorganism, being presented in Table 2 the specific culture medium for B. licheniformis; and in Table 3 the specific culture medium for B. subtilis with the addition of a stainless-steel balloon containing 5 L of the Endospore formation salt solution for the Bacillus spp. (Table 4), in Table 5 the specific culture medium for Lactobacillus and in Table 6 the culture medium for Pseudomonas and incubated for approximately 24-168 hours, with air flow 3.0-10.0 Nm³/h (=0.25-0.83 vvm) and temperature varying from 22-38° C., with exception of the species of Lactobacillus which are not incubated with aeration.

TABLE 2
CULTURE MEDIUM USED FOR THE GROWTH OF THE
B. LICHENIFORMIS FOR 200 L TANKS.

| | Reagents | |
|---|---|---|
| 01 | Maize | 2-20 g |
| 02 | Yeast extract | 1-10 g |
| 03 | NaCl | 1-10 g |
| 04 | Water | q.s.p. 1 L |

q.s.: quantum sufficit

TABLE 3
CULTURE MEDIUM USED FOR THE GROWTH OF
THE B. SUBTILIS FOR 200 L TANKS.

| | Reagents | | |
|---|---|---|---|
| 01 | Sodium glutamate | 5-20 | g |
| 02 | Peptone | 1-10 | g |
| 03 | KCl | 0.1-5 | g |
| 04 | MgSO₄•7H₂O | 0.1-2 | g |
| 05 | Yeast Extract | 0.1-5 | g |

| | | |
|---|---|---|
| 06 | Water | q.s.p. 1 L |

q.s.: quantum sufficit

TABLE 4
ENDOSPORE FORMATION SOLUTION FOR
THE TWO SPECIES OF BACILLUS.

| | Reagents | | |
|---|---|---|---|
| 01 | Ca (NO₃)₂ | 50-400 | g |
| 02 | MnCl₂ | 1.0-10 | g |
| 03 | FeSO₄ | 0.1-0.8 | g |

| | | |
|---|---|---|
| 04 | Water | q.s.p. 1 L |

q.s.: quantum sufficit

TABLE 5
CULTURE MEDIUM USED FOR THE GROWTH OF THE
LACTOBACILLUS SPP. UP TO THE SCALE OF 2000 L.

| | Reagents | | |
|---|---|---|---|
| 01 | K₂HPO₄ | 0.1-4 | g |
| 02 | Na₂HPO₄ | 2-10 | g |
| 03 | MgSO₄•7H₂O | 0.1-3 | g |
| 04 | NaCl | 0.1-1 | g |
| 05 | Yeast Extract | 10-35 | g |
| 06 | Peptone | 0.2-2 | g |
| 07 | KNO₃ | 0.1-1 | g |
| 08 | Soy flour | 5-20 | g |
| 09 | Saccharose | 5-20 | g |

| | | |
|---|---|---|
| 10 | Water | q.s.p. 1 L |

q.s.: quantum sufficit

TABLE 6
CULTURE MEDIUM USED FOR THE GROWTH OF THE
PSEUDOMONAS SPP. UP TO THE SCALE OF 180 L.

| | Reagents | | |
|---|---|---|---|
| 01 | K₂HPO₄ | 0.1-1 | g |
| 02 | KH₂PO₄ | 0.1-1 | g |
| 03 | MgSO₄•7H₂O | 0.1-3 | g |
| 04 | NaCl | 0.1-1 | g |
| 05 | Yeast Extract | 0.3-3 | g |
| 06 | (NH₄)₃PO₄ | 0.3-3 | g |
| 07 | KNO₃ | 0.5-5 | g |
| 08 | Solution MnSO₄ 10% | 0.05-0.1 | mL |
| 09 | Solution FeCl₃ | 0.05-0.1 | mL |
| 10 | Glycerol | 1-10 | mL |
| 11 | Saccharose | 1-10 | g |

| | | |
|---|---|---|
| 11 | Water | q.s.p. 1 L |

q.s.: quantum sufficit

### Example 2—Mix of Bacillus, Lactobacillus and Pseudomonas in Bioreactor

For the mix of the species of Bacillus, Lactobacillus and Pseudomonas in fermenter of 2,000 L, preferably there is used 1,200 L of the formulation of the Lactobacillus spp. (Table 5), which passes through a sterilization process for approximately 60 to 120 minutes, at a temperature of approximately 121° C. to approximately 130° C. Preferably, the sterilization is carried out at a pressure of approximately 1.0-2.0 Kgf/cm².

After the sterilization and cooling period, the tank containing the species of Lactobacillus spp. are then inoculated in the 2000 L fermenter, containing 1.200 L of the sterile cultivation medium, starting the fermenting process, which is of, preferably 24 to 72 hours at a temperature of 22° C.-38° C. The air flow is preferably of 1.0 Nm³/h to 2.5 Nm³/h (=0.0085-0.021 vvm). The pressure is preferably from 1.0 to 2.0 kgf/cm³. The shaking is preferably from 40 hz to 45 hz.

Preferably, after the fermentation time of the Lactobacillus spp., the mix of the tanks of B. licheniformis, B. subtilis and Pseudomonas spp. are inoculated and mixed to the 2.000 L fermenter. Preferably, the mixing time comprises from 30 to 120 minutes. Preferably, the product is bottled in gallons, in which packaging the product is stored.

### Example 3—the Induction of Metabolites Enables the Use in Industries for Phosphate Fertilizers and their by-Products

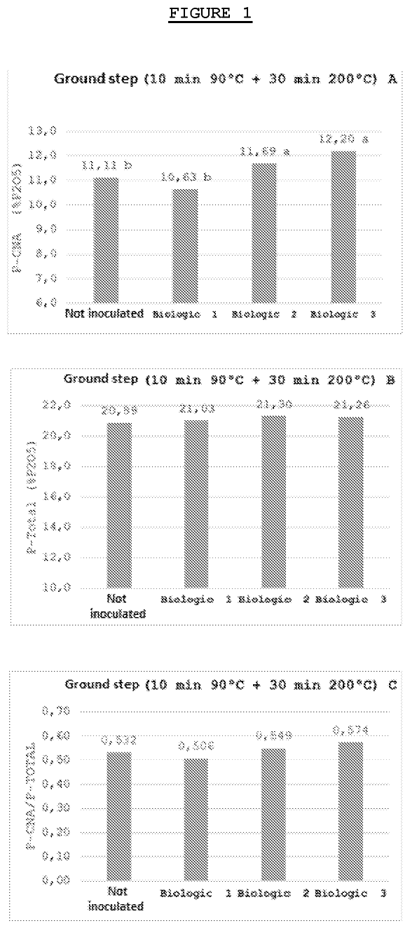

There were made applications of different combinations and proportions of microorganisms to verify the action in the phosphate fertilizers in different steps of the productive process of the industries of phosphate fertilizers. In FIG. 1, ground samples of the fertilizer were treated with the biologicals and passed through a process which simulates the production steps of the fertilizers, 90° C. for 10 minutes and 200° C. for 30 minutes. The treatment with the biological 4 (mix of Lactobacillus and Bacillus) presented higher conversion rate, increasing in 8% the availability of P relative to the control without the biological. The conversion rate is the P-CNA relation contained in the P-Total sample; the higher the value the better is the conversion rate, since in the extraction by the neutral citrate method there is simulated the absorption potential by the plant, while the P-total is all the phosphorus contained in the fertilizer.

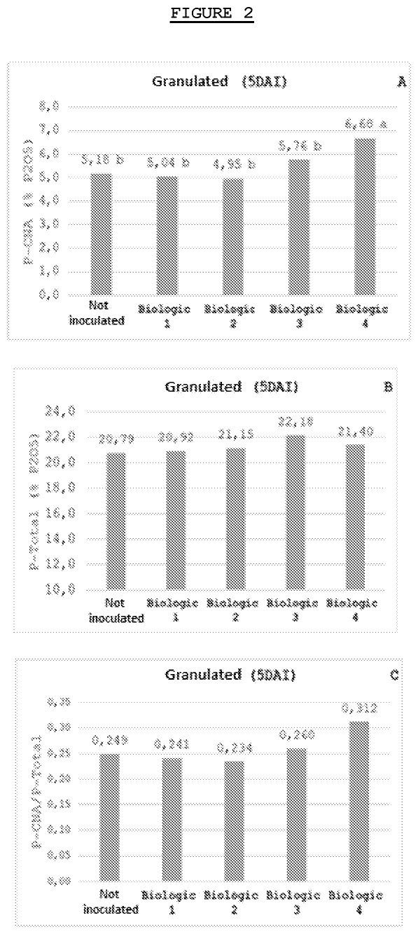

In the same manner, when the biologicals are applied to the granulated fertilizer (FIG. 2), another form of presentation of the product, there was an increase of 25% in the availability of P.

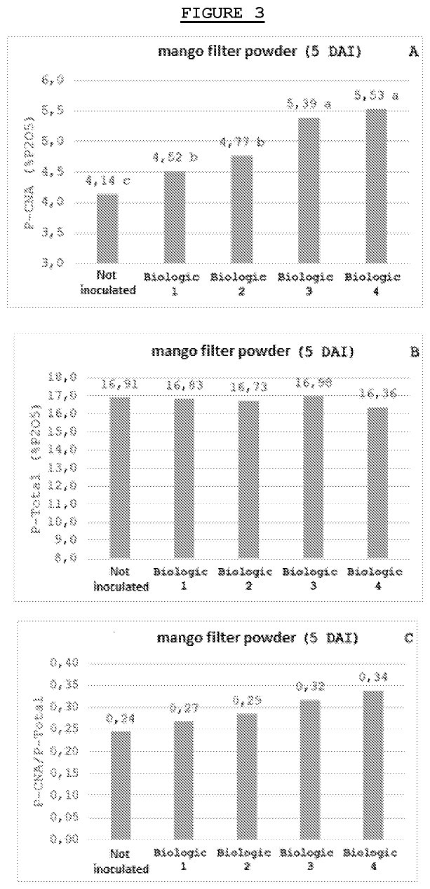

When the treatment is carried out in the by-product, which is generated during the industrial process for obtaining the fertilizer, the conversion rate was even more surprising in the treatments with the biologicals 3 (Lactobacillus and Pseudomonas) and 4 (FIG. 3), increasing in 30 and 38% the availability of P in the product, respectively.

Independent of the step of the productive process for obtaining the phosphate fertilizer, the use of the microorganisms results in higher availability of the phosphorus.

### Example 4—the Combination of the Microorganisms Enhances the Effect of the Phosphate Fertilizer

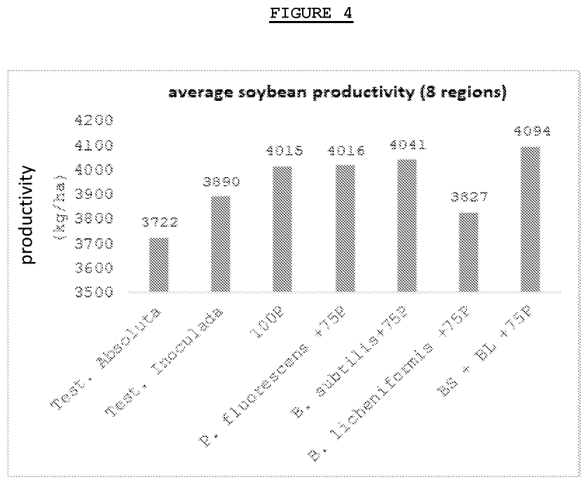

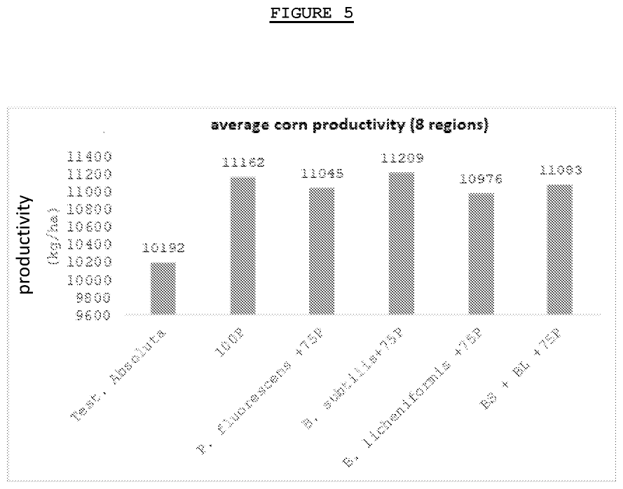

Field trials were carried out to validate the effect of the microorganisms in the soy and corn cultures with a reduction of 25% in the phosphate fertilization and the addition of the microorganism by itself or in a mixture. In FIG. 4, there is presented the average productivity of soy as from the 8 distinct regions, in a general manner the 25% reduction in fertilization did not result in loss of productivity when compared to the treatment with 100% of phosphate fertilization, except for the B. licheniformis by itself. It is possible to observe the potentiating effect on the availability of P which the microorganisms provide to the plants. In the same manner, when the trials were carried out for the corn culture (FIG. 5) the average productivity of 8 different edaphoclimatic areas with the application of the microorganisms on their own or in mixture provided a 25% reduction of the fertilization without resulting in loss of productivity. The greater productivity of both the cultures was only possible due to the use of the microorganisms which have the ability to make the P available to the plants, thus culminating in greater productivity. In this manner, the application of microorganisms in phosphate fertilizers, apart from acting in the greater availability of P in the fertilizers, also provided the residual effect in the field, since these microorganisms remain viable in the fertilizers, particularly the bacteria of the genus Bacillus which are industrially induced to the endospore formation, as mentioned in example 1.

Apart from the greater efficiency in the availability of P for the plants, the application of microorganisms in the cultures further presents greater availability of other nutrients such as, calcium (Ca), sulfur (S), copper (Cu) and iron (Fe) as presented in Table 7. It was possible to verify that the inoculation with the species of Bacillus presented greater absorption of the micronutrient Cu and Fe. These nutrients are essential for the plants, since they act as activator or component of enzymes, influence in the biological fixation of nitrogen, is essential to the balance of nutrients which regulate the plant transpiration, impacts in the photosynthesis and in the plant transpiration, among other benefits. Other advantages also with the application of the species of Pseudomonas is the availability of the macronutrients Ca and S, and the micronutrients Cu and Fe, presenting statistical difference when compared to the treatments with only 75% of P.

TABLE 7
ANALYSIS OF CHEMICAL ATTRIBUTES OF THE
AERIAL PART OF THE SOY PLANTS 35 DAE.

| | | Ca | | S | | Cu | | Fe |
|---|---|---|---|---|---|---|---|---|
| | Ca | Content | S | Content | Cu | Content | Fe | Content |
| Treatments | (g/Kg) | (g/pl) | (g/Kg) | (g/pl) | (mg/Kg) | (mg/pl) | (mg/Kg) | (mg/pl) |
| Witness | 10.99 b | 61.77 | 2.09 d | 11.75 | 13.25 b | 74.48 | 1203.50 c | 6764.67 |
| Inoculated | 11.36 b | 71.42 | 2.46 cd | 15.47 | 18.00 b | 113.17 | 2159.50 abc | 13576.66 |
| witness | | | | | | | | |
| 75P | 11.66 b | 67.65 | 2.62 bcd | 15.2 | 17.50 b | 101.53 | 2296.50 ab | 13324.17 |
| 100P | 11.02 b | 64.05 | 3.03 abc | 17.61 | 16.50 b | 95.9 | 2233.50 abc | 12981.6 |
| Pseudomonas + | 13.79 a | 84.93 | 3.75 a | 23.09 | 29.25 a | 180.14 | 3199.00 a | 19701.4 |
| Azospirillum | | | | | | | | |
| B. subtilis + B. | 10.21 b | 62.88 | 3.36 ab | 20.69 | 25.75 a | 158.58 | 3149.00 ab | 19393.47 |
| licheniformis | | | | | | | | |

LSD averages test (FISCHER) 5%.
DAE: days after the emergency.

1.-29. (canceled)

30. An industrial process for obtaining an agricultural composition employed in the manufacture and optimization of phosphate fertilizers having mineral, organomineral or organic origin, comprising:
(a) culturing followed by fermenting at least three microorganisms selected from the group consisting of Bacillus, Lactobacillus and Pseudomonas,
wherein each microorganism is cultured and fermented at a specific condition comprising one or more parameters selected from the group consisting of a temperature, an agitation speed, an aeration, a pressure and any combination thereof,
(b) inducing the biosynthesis of metabolites by the at least three microorganisms;
wherein the metabolites are capable of solubilizing and mineralizing insoluble phosphates in soluble phosphate compounds;
wherein each microorganism is fermented at a specific condition comprising one or more parameters selected from the group consisting of a temperature, an agitation speed, an aeration, a pressure and any combination thereof to biosynthesize the metabolites; and
(c) admixing the at least three microorganisms and the biosynthesized metabolites and bottling the agricultural composition,
wherein the biosynthesized metabolites by Pseudomonas and Lactobacillus can act instantaneously on inorganic phosphates during the manufacturing process of the phosphate fertilizers, and wherein the biosynthesized metabolites by Bacillus act on inorganic phosphates applied to an agricultural field.

31. The industrial process of claim 30, wherein the induction of the biosynthesis of metabolites of step (b) by Bacillus and Pseudomonas occurs at a temperature of about 22° C.-38° C. and an air flow of about 3.0-10.0 Nm³/h or about 0.25-about 0.83 vvm in a tank; and the inducing of the biosynthesis of metabolites by Lactobacillus occurs at a temperature of about 22° C.-38° C. and an air flow of about 3.0-about 10.0 Nm³/h or about 0.25-about 0.83 vvm in a fermenter.

32. The industrial process of claim 30, wherein culturing of step (a) of the three microorganisms comprises inoculating each microorganism into a separate culture.

33. The industrial process of claim 30, wherein culturing of the three microorganisms comprises expanding each culture in a volume from 100 mL to 1 L, then to 10 L, then to 180 L, and then up to 2000 L.

34. The industrial process of claim 30, wherein the culturing of step (a) of the Bacillus and the Pseudomonas in a culture medium volume ranging from 1 L-10 L is by agitation in an orbital shaker at about 80-about 200 rpm for about 8 hours-about 48 hours, wherein the 1 L culture medium is in a glass flask and the 10 L culture medium is in a stainless-steel balloon.

35. The industrial process of claim 34, wherein the culturing of the Bacillus is with an airflow of about 0.25-about 1.0 Nm³/h or about 4.16-about 16.67 vvm for a 1 L culture; and wherein the serially expanding of the Bacillus and Pseudomonas is with an airflow of about 0.25-about 1.5 Nm³/h or about 0.41-about 1.67 vvm for a 10 L of culture.

36. The industrial process of claim 34, wherein two stainless-steel balloons both comprising 10 L of culture medium and either Bacillus, Pseudomonas or Lactobacillus are inoculated into a fermenter tank comprising 180 L of culture medium.

37. The industrial process of claim 36, wherein the culturing of step (a) of either Bacillus, Pseudomonas or Lactobacillus in a fermenter tank comprising 180 L of culture medium is for about 24-about 168 hours.

38. The industrial process of claim 37, wherein the culturing of step (a) of the Bacillus or Pseudomonas is with an airflow of about 1.0-about 15.0 Nm³/h or about 0.16-about 0.25 vvm.

39. The industrial process of claim 34, wherein the culturing of step (a) of the Lactobacillus is at a temperature of about 22° C.-about 38° C. and without agitation or aeration.

40. The industrial process of claim 30, wherein the fermenting of step (a) is by batch fermentation.

41. The industrial process of claim 30, wherein the fermenting of step (a) is at a pressure ranging from about 1.0-about 2.0 kgf/cm² in a 2000 L fermenter.

42. The industrial process of claim 30, wherein the fermenting of step (a) is with agitation of about 40 hz-about 45 hz, at a temperature of about 22° C.-about 30° C., and with an airflow of about 1.0-about 2.5 Nm³/h or about 0.0085-about 0.021 vvm.

43. The industrial process of claim 30, wherein the admixing is in a 2000 L fermenter for about 30-about 120 minutes.

44. A method for using the agricultural composition of claim 30 selected from the group consisting of: fertilizer production, conversion of by-products generated in the industrialization of fertilizers into compounds with agricultural applications, broadcasting and sowing furrow together with a phosphate fertilizer; directly applying to seeds or a sowing furrow; and increasing the availability of macronutrients and micronutrients in the plants with agricultural interest.
